(19) United States
(12) Patent Application Publication
(10) Pub. No.: US 2005/0255131 A1
(43) Pub. Date: Nov. 17, 2005

(54) Clindamycin compositions and delivery system therefor

(76) Inventors: Mohan Vishnupad, Easton, CT (US); Naomi Vishnupad, Reading, MA (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/124,446

(22) Filed: May 6, 2005

Related U.S. Application Data
(60) Provisional application No. 60/569,808, filed on May 11, 2004.

(51) Int. Cl. A61K 31/704; A61K 9/70

(52) U.S. Cl. 424/400; 514/35

(57) ABSTRACT

Clindamycin compositions contain water and a co-solvent and optionally an emulsifier.

### RELATED APPLICATIONS

This application claims priority to U.S. Provisional Application Ser. No. 60/569,808 filed on May 11, 2004, the entire disclosure of which is incorporated herein by this reference.

### BACKGROUND

The combination of benzoyl peroxide (BPO) and antibiotics, such as clindamycin phosphate, has been described in many journal articles and several patents. These two incompatible active ingredients degrade upon aging at elevated temperatures. This makes it extremely difficult to comply with the FDA requirements and have a combination product that has a 2-year shelf life.

One brand name product that contains BPO and clindamycin phosphate is BENZACLIN® (available from Dermik Laboratories, Berwyn, Pa.). BENZACLIN is provided to pharmacies as a kit (with the active ingredients in separate containers) and compounding instructions. The kit includes a vial containing clindamycin powder and a separate container containing a BPO gel. To fill a prescription, the pharmacist dissolves the clindamycin salt by adding a measured amount of water to the vial containing the clindamycin powder. The clindamycin solution is then poured into the jar containing the BPO gel, and the contents are mixed with a spatula for uniform blending. This pharmacy-compounded, three component product has an expiration date of three months at room temperature.

Another brand name product that contains BPO and clindamycin phosphate is DUAC® (available from Stiefel Laboratories, Inc., Coral Gables, Fla.). DUAC has the same combination of BPO and clindamycin phosphate in a specific gel formulation mixed together. Due to the incompatibility of the two actives at elevated temperatures, the combined product requires refrigeration. Thus, once made, DUAC must be refrigerated at the production plant, during shipment, and in the pharmacy's inventory until the product is dispensed to a consumer. Once dispensed, DUAC has a 60 day expiration date at room temperature. Refrigeration of the inventory and during shipment is quite expensive and substantially drives up the cost of the product.

Clindamycin salts, such as clindamycin phosphate, are commonly used in aqueous solutions. However, the chemical stability of aqueous clindamycin solutions is limited at both low temperatures and elevated temperatures. For example, U.S. Pat. No. 6,117,843 discloses a two-component kit containing an aqueous solution of clindamycin and an aqueous benzoyl peroxide suspension wherein the aqueous solution of clindamycin has a clindamycin concentration in the range from 2% to 15% by weight and a pH within a range from 3.5 to 7, preferably within a range from 6 to 6.5, in order to inhibit precipitation of the clindamycin from the solution, particularly when the solution is exposed to cold temperatures during storage.

Recently, technologies for overcoming such incompatibility problems have been described in U.S. Pat. No. 6,462,025 and U.S. Application No. 2002/0193321A1. These technologies remove the need for pharmacy compounding and expensive inventory refrigeration. Using a dual pump dispenser, which keeps two active drug formulations separate during shipment and in inventory, the consumer can simultaneously dispense metered doses of the two active formulations and blend them in the hand upon use. While these technologies satisfactorily provide the full two year expiration date, there exists room for improvement in the area of packaging, storing and dispensing compositions containing incompatible active ingredients such as BPO and clindamycin phosphate.

### SUMMARY

The present clear solution compositions contain a solution of water, clindamycin and glycerin, wherein the amount of water is insufficient alone to solubilize the clindamycin. In some embodiments, the compositions contain water, clindamycin phosphate and glycerin, the weight ratio of clindamycin phosphate to water being from about 1:1.5 to about 1:16.5, and optionally a water soluble emulsifier. In some embodiments, the present compositions contain water, clindamycin in an amount greater than 6% by weight based on the weight of the water, and a co-solvent in which clindamycin is not soluble, the co-solvent being present in an amount sufficient to solubilize the clindamycin in the water. The co-solvent may be glycerin. The compositions optionally contain an emulsifier. In particularly useful embodiments, the compositions contain water in an amount of about 25% to about 35% by weight of the composition, glycerin in an amount of about 65% to about 75% by weight of the composition, clindamycin in an amount of about 1.5 to about 10% by weight of the composition; and optionally a water soluble emulsifier.

In another aspect, an apparatus is provided which includes a first chamber containing a first composition containing clindamycin, water, a co-solvent such as glycerin and optionally an emulsifier, a second chamber containing a second composition that contains benzoyl peroxide, and at least one outlet for dispensing the first and second compositions.

In another aspect, a method is provided which includes providing a first composition, the first composition containing clindamycin, water, a co-solvent such as glycerin and optionally an emulsifier; providing a second composition comprising benzoyl peroxide; storing the first and second compositions separately from each other in first and second chambers, respectively of an apparatus comprising first and second chambers; mixing the first and second compositions within the apparatus; and dispensing the first and second compositions.

### BRIEF DESCRIPTION OF THE DRAWINGS

Various embodiments of the present disclosure will be described herein below with reference to the figures wherein:

FIG. 1 is a vertical cross-sectional of a chamber in chamber single pump dispenser.

FIG. 2 is a vertical cross-sectional view showing the configuration of the chamber in chamber single pump dispenser of FIG. 1 when the contents of the two chambers are being mixed prior to use.

FIG. 3 is a vertical cross-sectional view showing the configuration of the chamber in chamber single pump dispenser of FIG. 1 at the time of dispensing.

### DETAILED DESCRIPTION

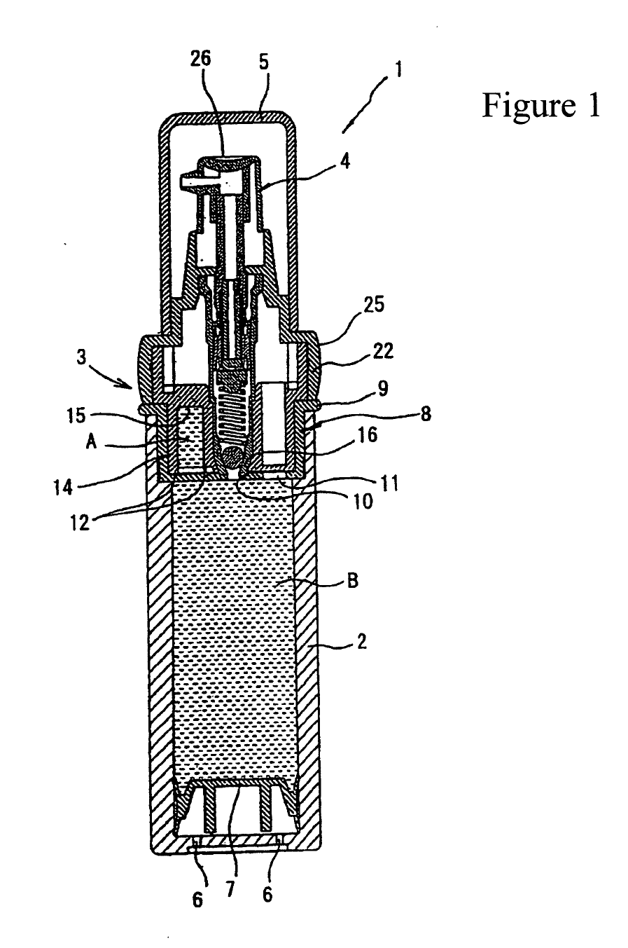

By utilizing a chamber in a chamber single pump delivery system of the type disclosed in WO 03/082703A1 and EP1498362A1, the disclosures of which are incorporated herein in their entirety, the elevated temperature drug incompatibility may be overcome. As seen in FIG. 1, the small chamber 14, which has a small capacity, contains a composition (A). In accordance with embodiments of the present disclosure, Composition (A) may contain one of the active ingredients, such as antibiotic solution, or antibiotic powder blend. This chamber 14 is inserted into a main chamber 2, which contains a composition (B). In accordance with embodiments of the present disclosure, Composition (B) may contain the other active ingredient, such as a benzoyl peroxide suspension or emulsion. The ratio of both actives is calculated in accordance with embodiments of the present disclosure for this system to deliver the combination of BPO and antibiotics at a concentration that has already been established commercially and is approved by the FDA. The small chamber 14 is locked inside the main chamber 2. The two active drugs never come in contact with each other until the consumer activates the system before use.

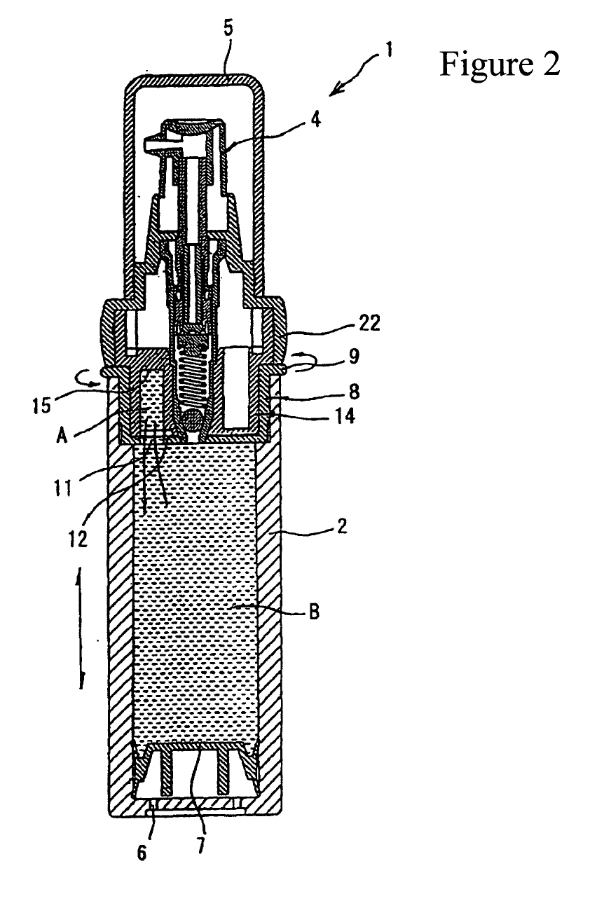

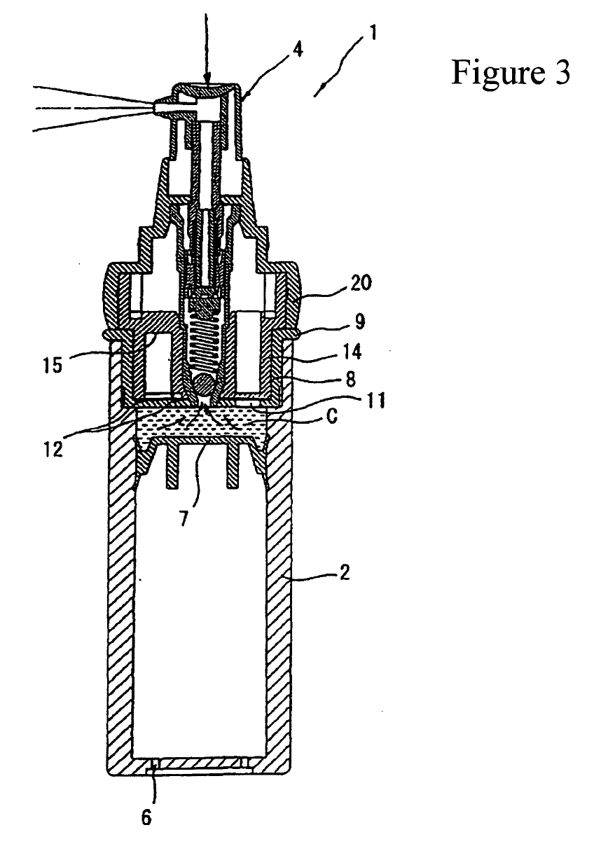

As shown in FIG. 2, the user rotates housing 2 relative to periphery section 25 of supporting tube 22 to release the composition from the small chamber 14 into the main chamber 2 through the mixing-communication hole 11. The user may then shake the package to blend both products to generate mixed contents (C) (see FIG. 3). In accordance with embodiments of the present disclosure, Compositions (A) and (B) may be specially formulated with low viscosity to facilitate quick and uniform blending. The user can then use the pump delivery mechanism (as shown in FIG. 3) to dispense the mixed contents (C). In accordance with embodiments of the present disclosure, mixed contents (C) contains the powder or solution of composition (A) blended with the suspension or emulsion of composition (B) to deliver the combination of both actives for treating the skin. The shelf life or expiration date for such products, from the time of manufacturing to the time of patient's total consumption of the dispensed product, may exceed two years. This expiration date is economical for the marketer and desirable for the FDA.

The small chamber has a limited capacity. The active concentrations in both chambers should be accurately balanced and maintained in order to deliver the desirable concentrations for product efficacy and FDA compliance. To achieve a desirable concentration balance, a higher concentration of antibiotics in the small chamber, which when diluted with the main chamber containing BPO, provides the desirable concentrations for skin application.

For the clindamycin phosphate solution, a maximum of about 6% salt is soluble in water. This solution is not stable upon long-term storage at elevated temperatures. In order to utilize the chamber in a chamber delivery system wherein the contents of a small chamber are combined with the contents of a larger chamber, compositions containing a concentration of clindamycin phosphate higher than 6% are desirable. In accordance with the present disclosure, solutions containing concentrations of clindamycin in excess of 6% in a minimum amount of water may be prepared by using co-solvents such as glycerin. Clindamycin phosphate by itself is not soluble in glycerin alone. Surprisingly, however, by using a small amount of water, glycerin solubilizes clindamycin salt. Thus, the present compositions may contain a solution of water, clindamycin and glycerin, wherein the amount of water is insufficient alone to solubilize the clindamycin. The term “solubilize” as used with respect to solubilizing clindamycin refers to the ability to dissolve clindamycin and optionally with heating and stirring and maintaining clindamycin in solution at room temperature for at least 24 hours. Clindamycin phosphate solutions prepared in accordance with the present disclosure using water and glycerin as a co-solvent do not precipitate upon long-term storage at room temperature or at cold temperatures of, for example, about 2 to about 10° C.

By increasing the concentration of clindamycin in solution by means of a co-solvent such as glycerin, the present methods make it possible to dissolve the salt in a small amount of water. The ratio of water to co-solvent can be from about In accordance with the present methods, 1 gram of clindamycin phosphate dissolves in 4.2 grams of water (24% solution) by using glycerin as a co-solvent. Due to the limited solubility of clindamycin phosphate in water, this concentration is not soluble in water alone. In particularly useful embodiments, the co-solvent system includes at least 25% water, with the balance being glycerin. Glycerin acts as a co-solvent, rendering a clear solution. For the chamber in chamber system the small chamber can contain, for example, 5 grams of the unique 6.05% clindamycin solution, delivering 1.00% of active clindamycin in the final blended product.

Composition (B) can be any benzoyl-peroxide containing composition. For example, the composition (B) can be an aqueous emulsion or suspension containing benzoyl peroxide. Formulations for benzoyl-peroxide containing aqueous emulsions and suspensions can be readily formulated by those skilled in the art and typically contain benzoyl peroxide in an amount from about 1% to about 20%, water in an amount from about 5% to about 80%.

Composition (B) can also be a substantially anhydrous benzoyl-peroxide containing composition. Illustrative examples of substantially anhydrous benzoyl-peroxide containing compositions are described, for example, in U.S. Pat. No. 5,632,996, the entire disclosure of which is incorporated herein by this reference.

A variety of optional components such as, for example, humectants, emollients, dyes, medicaments, texture modifiers, fillers and the like may be included in composition (A) or composition (B) or both.

Depending on the particular clindamycin and the BPO compositions employed, the distribution in the mixed contents (C) may not be uniform, resulting in a non functional product. For example, when a clindamycin solution containing only glycerin and water is released from small chamber 14 into certain BPO compositions and shaken, the blending together of the actives may not be uniform. Thus, an emulsifier may optionally be included in composition (A) or composition (B) or both to ensure uniform blending of the compositions when generating mixed contents (C). The present compositions may contain from about 0.01% to about 10%, more typically from about 0.1% to about 5%, of emulsifier, based on the weight of the composition. Any emulsifier may be used. The emulsifier may be nonionic, anionic or cationic. Suitable emulsifiers are disclosed, for example, in McCutcheon's Detergents and Emulsifiers, North American Edition, pages 317-324 (1986), the disclosure of which is incorporated herein in its entirety by this reference. Suitable emulsifiers include, but are not limited to ethoxylated fatty acids, ethoxylated esters, phosphated esters, polyoxyethylene fatty ether phosphates, fatty acid amides, acyl lactylates, soaps and mixtures thereof. Non-limiting examples of such emulsifiers include polyoxyethylene (8) stearate, myristyl ethoxy (3) myristate, polyoxyethylene (100) monostearate, lauric diethanolamide, stearic monoethanolamide, hydrogenated vegetable glycerides, sodium stearoyl-2-lactylate, calcium stearoyl-2-lactylate. Soaps are also acceptable emulsifiers. The soaps may be alkali metal or triethanolamine salts of long chain fatty acids. Such soaps include sodium stearate, triethanolamine stearate and the similar salts of lanolin fatty acids. In certain embodiments, emulsifying surfactants having an HLB value from about 3 to below 12 such as steareth-2, PEG-5 soya sterol oil, PEG-10 soya sterol oil, diethanolamine cetyl phosphate, sorbitan monostearate (SPAN 60), diethyleneglycol monostearate, glyceryl monostearate, and mixtures thereof can be used. Alternatively, emulsifying surfactants can be used that have an HLB value of 12 or above (or about 12 and above) such as Steareth-21, polyoxyethylene sorbitan tristearate (TWEEN 65), polyethylene glycol 20 sorbitan monostearate, polyethylene glycol 60 sorbitan monostearate, polyethylene glycol 80 sorbitan monostearate, Steareth-20, Ceteth-20, PEG-100 stearate, sodium stearoyl sarcosinate, hydrogenated lecithin, sodium cocoylglyceryl sulfate, sodium stearyl sulfate, sodium stearoyl lactylate, PEG-20 methyl glucoside sesquistearate, PEG-20 glyceryl monostearate, sucrose monostearate, sucrose polystearates (having a high proportion of sucrose monostearate), polyglyceryl 10 stearate, polyglyceryl 10 myristate, Steareth-10, DEA oleth 3 phosphate, DEA oleth 10 phosphate, PPG-5 Ceteth 10 phosphate sodium salt, PPG-5 Ceteth 10 phosphate potassium salt, and mixtures thereof. It should be understood that combinations of emulsifiers can be employed. For example, the present compositions may include an emulsifying surfactant having an HLB value about 12 or below and an emulsifying surfactant having an HLB value of about 12 or above. “HLB” is well known to one of ordinary skill in the art and means hydrophobic lipophilic balance. See, “The HLB System, A Time-Saving Guide to Emulsifier Selection,” ICI Americas Inc., August (1984) the disclosure of which is incorporated herein by reference in its entirety. In certain embodiments, polysorbate is added as an emulsifier to the clindamycin/glycerin/water solution in the small chamber. Suitable polysorbates are commercially available under the trqdename TWEEN®, such as TWEEN 20® or TWEEN 8®. Upon releasing this solution from the small chamber into the larger chamber containing the BPO suspension or emulsion, and shaking the bottle, the actives may advantageously be uniformly blended.

The weight ratio of composition (A) to composition (B) can be from about 1:2 to about 1:20, typically from about 1:3 to about 1:10. In particularly useful embodiments, the weight ratio of composition (A) to composition (B) can be from about 1:4.

In order that those skilled in the art may be better able to practice the compositions and methods described herein, the following examples are given as an illustration of the preparation of the present dispensing compositions and system. It should be noted that the invention is not limited to the specific details embodied in the examples.

### EXAMPLE 1

The inner, smaller chamber of a chamber in a chamber package is filled with clindamycin phosphate composition and the larger, main chamber is filled with a BPO suspension. The formulation for each composition is as follows:

Small, inner chamber (Composition A—clindamycin phosphate solution)

| Ingredient | % |
|---|---|
| Clindamycin PO₄ (82.7%) | 6.05 |
| H₂O | 25.00 |
| Glycerin 99% | 68.95 |

Main Chamber (Composition B—Benzoyl Peroxide Suspension)

| Ingredient | % |
|---|---|
| BPO 75% | 8.75 |
| C₁₂₋₁₅ alkyl benzoate | 8.00 |
| Water | 13.75 |
| Glycerin | 8.50 |
| Propylene glycol | 10.50 |
| Volatile silicone | 35.00 |
| Dry flo starch | 15.00 |
| Tween 20 | 0.50 |

Ratio of Composition A and B

| | |
|---|---|
| Composition A | 20.00% |
| Composition B | 80.00% |

Final Concentration of Actives in the Blend:
Clindamycin: 1.00%
BPO: 5.00%

### EXAMPLE 2

The inner, smaller chamber of a chamber in a chamber package is filled with clindamycin phosphate composition and the larger, main chamber is filled with a BPO emulsion. The formulation for each composition is as follows: Composition A: clindamycin solution

| Ingredient | % |
|---|---|
| Clindamycin PO₄ (82.7%) | 6.05 |
| Water | 25.00 |
| Glycerin 99% | 68.95 |

Composition B: BPO Emulsion

| Ingredient | % |
|---|---|
| carbopol 980 | 0.25 |
| disodium EDTA | 0.50 |
| Brij 721 | 1.12 |
| Brij 72 | 1.52 |
| cetyl stearyl alcohol | 1.52 |
| Arlacel 165 | 1.52 |
| Emulsifier 10 | 1.00 |
| Tween 20 | 1.25 |
| BPO 75% | 8.34 |
| Alkyl benzoate (FINSOLV TN ®) | 7.00 |
| DiH₂O | 75.98 |

Ratio of Composition A and B

| | |
|---|---|
| Composition A | 20.00% |
| Composition B | 80.00% |

Final Concentration of Actives in the Blend:
Clindamycin: 1.00%
BPO: 5.00%

### EXAMPLE 3

The inner, smaller chamber of a chamber in a chamber package is filled with clindamycin phosphate composition and the larger, main chamber is filled with a BPO suspension. The formulation for each composition is as follows:

Small inner chamber (Composition A: Clindamycin solution)

| Ingredient | % |
|---|---|
| Clindamycin PO₄ (100%) | 5.00 |
| Water | 25.00 |
| Polysorbate 20 | 0.65 |
| Glycerin 99% | 69.35 |

Composition B: BPO Emulsion

| Ingredient | % |
|---|---|
| Carbopol 980 | 0.25 |
| Glycerin | 3.00 |
| Disodium EDTA | 0.30 |
| Brij 721 | 1.12 |
| Brij 72 | 1.52 |
| Arlacel 165 | 1.52 |
| Emulsifier 10 | 1.00 |
| cetyl stearyl alcohol | 1.52 |
| Lanolin oil | 0.47 |
| Polysorbate 20 | 1.25 |
| BPO 75% | 8.34 |
| Alkyl benzoate (FINSOLV TN ®) | 7.00 |
| Water | 72.71 |

Ratio of Composition A and B

| | |
|---|---|
| Composition A | 20.00% |
| Composition B | 80.00% |

Final Concentration of Actives in the Blend:
Clindamycin: 1.00%
BPO: 5.00%

Similarly, a high concentration clindamycin powder blend can be used in the small chamber and blended with the BPO emulsion or suspension. The concentration of the high concentration clindamycin powder blend is calculated to achieve the 1.0% clindamycin and 5.0% BPO in the final blended composition.

The chamber in a chamber delivery system is also desirable to formulate water incompatible compounds such as ascorbic acid, green tea extract, hydroquinone, 2,4,6-cycloheptraxien-1-one, w-hydroxy-4-(1-methyethyl) or other skin bleaching agents, that discolor in the presence of light, heat, and oxidation. By keeping such compounds separate from aqueous media, the temperature effect on the blended product (which is exposed only to room temperature conditions under normal consumer usage), is eliminated.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A composition comprising:
a solution of water, clindamycin and glycerin, wherein the amount of water is insufficient alone to solubilize the clindamycin; and
optionally a water soluble emulsifier.

2. A composition as in claim 1 wherein clindamycin is present in an amount of at least about 2 to about 10% by weight of the composition.

3. A composition as in claim 1 wherein an emulsifier is present in an amount of at least about 0.01 to about 1% by weight of the composition.

4. A composition as in claim 1 comprising a polysorbate as an emulsifier.

5. An apparatus comprising:
a first chamber containing a first composition, the first composition being a composition of claim 1;
a second chamber containing a second composition comprising benzoyl peroxide; and
at least one outlet for dispensing the first and second compositions.

6. An apparatus as in claim 5 wherein the second composition is an aqueous emulsion or suspension.

7. An apparatus as in claim 5 wherein the volume ratio of the first composition to the second composition is from about 1:2 to about 1:20.

8. An apparatus as in claim 5 wherein the volume ratio of the first composition to the second composition is from about 1:4.

9. An apparatus as in claim 5 wherein the first and second chambers can be selectively moved to a first position wherein the contents of the first and second chambers are isolated and a second position wherein the contents of the first and second chambers can be mixed.

10. A composition comprising:
water;
clindamycin phosphate in an amount greater than 6% by weight based on the weight of the water; and
glycerin in an amount sufficient to solubilize the clindamycin,
wherein the composition is a clear solution.

11. A composition as in claim 10 wherein glycerin is present in an amount from about 65 to about 85 present by weight of the total composition.

12. A composition as in claim 10 further comprising an emulsifier.

13. A composition as in claim 12 wherein the emulsifier is present in an amount from about 0.01 to about 1% by weight of the total composition.

14. A composition as in claim 12 wherein the emulsifier is a polysorbate.

15. A method comprising:
providing a first composition, the first composition being a composition of claim 1;
providing a second composition comprising benzoyl peroxide;
storing the first and second compositions separately from each other in first and second chambers, respectively of an apparatus comprising first and second chambers;
mixing the first and second compositions within the apparatus; and
dispensing the first and second compositions.

16. A method comprising:
providing a first composition, the first composition being a composition of claim 10;
providing a second composition comprising benzoyl peroxide;
storing the first and second compositions separately from each other in first and second chambers, respectively of an apparatus comprising first and second chambers;
mixing the first and second compositions within the apparatus; and
dispensing the first and second compositions.

17. A composition comprising:
water, clindamycin phosphate and glycerin, the weight ratio of clindamycin to water being from about 1:1.5 to about 1:16.5.
